(19) United States
(12) Patent Application Publication
(10) Pub. No.: US 2021/0162096 A1
(43) Pub. Date: Jun. 3, 2021

(54) MATRICES FOR TISSUE ENGINEERING IN THE FORM OF FOAMS, FIBRES AND/OR MEMBRANES FORMED OF POLYMERS, CERAMICS, POLYMERIC COMPOSITES AND/OR CERAMIC COMPOSITES CONTAINING BIXA ORELLANA L. EXTRACT AND METHOD OF PRODUCTION

(71) Applicant: Centro Federal de Educacao Tecnologica de Minas Gerais, Belo Horizonte (BR)

(72) Inventors: Roberta VIANA FERREIRA, Lagoa Santa (BR); Aline BRUNA DA SILVA, Belo Horizonte (BR); Ana ELISA ANTUNES DOS SANT, Belo Horizonte (BR); Guilherme HOFFMAN DE CASTRO, Belo Horizonte (BR); Fernanda VIEIRA DE SA, Belo Horizonte (BR); Francisco VIEIRA DOS SANTOS, Belo Horizonte (BR)

(21) Appl. No.: 16/641,443

(22) PCT Filed: Jun. 14, 2018
(86) PCT No.: PCT/BR2018/050193

§ 371 Date: Feb. 8, 2021

(30) Foreign Application Priority Data
Aug. 23, 2017 (BR) BR 102017018080-8

(51) Int. Cl. A61L 27/14; A61L 27/10; A61L 27/40; A61L 27/56

(52) U.S. Cl. CPC A61L 27/14; A61L 27/56; A61L 27/40; A61L 27/10

(57) ABSTRACT

The present invention relates to matrices for tissue engineering in the form of foams, fibres and/or membranes formed of polymers, ceramics, polymeric composites and/or ceramic composites containing Bixa orellana L. extract capable of inducing tissue regeneration in vivo and in vitro, preventing inflammatory processes and fungal and bacterial contamination during processes of regeneration. The matrices can be two-dimensional or three-dimensional, and have the morphology, porosity and pore size required for tissue growth and regeneration, said structure being particularly suitable for tissue growth in vitro or tissue regeneration in vivo, and the regeneration of hard or soft tissue. Methods for producing said matrices are also described, which include impregnating the materials with the extract and subsequently processing the scaffolds in the form of foams, fibres or membranes, which can be carried out by electro spinning, producing foams by leaching particles, the method of foaming, lyophilization or casting.

### CROSS REFERENCE TO RELATED APPLICATION

This Application is a 371 of PCT/BR2018/050193 filed on Jun. 14, 2018 which, in turn, claimed the priority of Brazilian Patent Application No. BR102017018080-8 filed on Aug. 23, 2017, both applications are incorporated herein by reference

### BACKGROUND OF THE INVENTION

The present invention relates to tissue engineering matrices in the form of foams, fibers and/or membranes consisting of polymers, ceramics, polymeric composites and/or ceramic composites containing Bixa orellana L. extract capable of inducing tissue regeneration in vivo , tissue growth in vitro and the production method. Tissue Engineering is a multidisciplinary study field that applies the principles of Engineering and life sciences for biological substitutes development, aiming the restoration, maintenance or improvement of tissue function (LANGER and VACANTI, 2015). It is defined as a science that seeks the development and manipulation of bioactive molecules, cells, tissues, or organs grown in the laboratory to replace or support the function of defective or damaged body parts.

The need to replace or regenerate a part of the human organism is frequent and constant due to: degenerative diseases that arise with age, fractures, human body disfiguration in accidents with vehicles, weapons, tools and sports, or congenital deficiencies. The alternatives currently consist of replacing damaged parts with organ implants or transplants. However, a third of skeletal system prostheses and heart valves fail within 10 to 20 years requiring revision surgery. In addition, biomaterials can be considered an adjustment, since there is no man-made material capable of responding as a human tissue to changes in physiological loading and biochemical stimuli—wear/failure. Organ transplants also involve a number of disadvantages such as the need for lifelong immune suppression and the scarcity of organ numbers. In this context, Tissue Engineering proposes to produce living and functional organs and tissues in laboratory, using patient cells and biodegradable materials, the scaffolds.

Currently, tissue engineering has excelled in applications involving tissue development in vitro for application in toxicity tests and effectiveness for new drugs and cosmetics development, and as models for new treatments studies for diseases such as cancer.

Tissue Engineering uses biological elements, such as cells, biocompatible and biodegradable materials called scaffolds, bioactive molecules and bioreactors to achieve the proposed objective (PATEL, BONDE and SRINIVASAN, 2011). The construction or regeneration of tissues using the concept of Tissue Engineering consists of cultivating cells in a structure with morphology and composition similar to the extracellular matrix of the tissue in the presence of bioactive substances. Basically a small number of cells are harvested from the patient using a biopsy technique and then cultured in the laboratory. The cells are expanded into a three-dimensional or fibrous, natural or synthetic matrix (biomaterial—scaffold), in the presence of bioactive molecules (growth and differentiation factors, and drugs). In the presence of stimuli that simulate the physiological condition, cells will secrete the extracellular matrix components to create a new living tissue that can be used as a substitute for implantation in the defective site in the patient. The matrix degrades and at the end of the process there is only a new tissue or organ composed of extracellular matrix and cells. In the case of using the patient's cells, there is no immune rejection response to the implanted tissue, eliminating the requirement for the use of immunosuppressants.

In the context of Tissue Engineering, scaffolds can be defined as the three-dimensional structure or matrix that provides temporary mechanical support for cells during cell adhesion and proliferation phenomena, in other words, the scaffold simulates the extracellular matrix. (MURPHY and MIKOS, 2007) Therefore, the scaffold's function is to mechanically support, define the geometry of the tissue to be formed, provide a biochemical environment conducive to cell growth and degrade when the tissue growth or regeneration process is completed. Therefore, the chemical composition, physical structure and biological functionality are important attributes for the biomaterials used in scaffolds manufacturing (MA, 2008). In summary, scaffolds are used in the following purposes: to allow cellular connection and migration, delivery and retention of biochemical factors, to allow diffusion of vital nutrients to cells and products and to exercise a certain mechanical and biological influence to guide the behavior of cells. (PATEL, BONDE and SRINIVASAN, 2011)

The internal structure of the scaffolds is extremely important for this component performance. The porosity and pore size of these matrices have direct implications for functionality in biomedical applications. Open pores and interconnection networks are essential for nutrition, proliferation and cell migration for tissue vascularization and formation of new tissues. A porous surface also facilitates the interconnection between the scaffolds and the surrounding tissue, improving the stability of the implant. Thus, the structure of porous networks contributes to the promotion of tissue formation. Structures composed of non-woven micro or nanofiber layers are also very advantageous for application as scaffolds as they have unique characteristics such as high surface area, high porosity, possibility of surface modification and may have mechanical properties compatible with that of the extracellular matrix, characteristics that combined mimic the topography of the extracellular matrix and facilitate cell adhesion, migration and proliferation (ref.1 in annex).

Various materials have been used as scaffolds in tissue regeneration: metals, ceramics, descellularized extracellular matrices, polymers, both natural and synthetic, and polymer/ceramic composites.

Polymers have high flexibility in terms of design, as the chemical composition and structure can be changed according to the tissue needs (MA, 2008). The materials that stand out for the production of scaffolds include synthetic and natural biodegradable polymers. (PATEL, BONDE and SRINIVASAN, 2011) The scaffolds produced from natural polymers are interesting because they have familiar chemical structures for cellular recognition, providing natural mechanisms for tissue remodeling. However, these natural polymers are very susceptible to structural changes during the scaffold purification and manufacturing processes. (LUO, ENGELMAYR, et al., 2007). An example of this is collagen. This natural macromolecule is one of the most used today in the production of reconstructed skin and cartilage in the laboratory. However, scaffolds obtained from collagen have a high cost due to difficulties in processing and controlling the physical and chemical properties of this material. Other polymers that stand out in this area include gelatin, fibrin, fibronectin, chitosan, starch, alginate and hyaluronic acid.

The scaffolds produced from synthetic polymers offer a variety of mechanisms for adjusting cellular behavior, in addition to providing the formation of blends that allow the control of the mechanical behavior and degradability of the material (PATEL, BONDE and SRINIVASAN, 2011; LUO, ENGELMAYR, et al., 2007). Among the synthetic polymers most applied in Tissue Engineering there are polyesters such as poly (glycolic acid), poly (lactic acid) and their copolymers, poly (butylene succinate), polyfumarates, polyhydroxyalkanoates, poly (glycerol sebacate), poly (acrylic acid), polyurethanes, pluronic F-127, poly (orthoesters), polyphosphazenes, polyanhydrides, polypyrroles and semi-synthetic cellulose derivatives.

The use of synthetic or semi-synthetic polymers in the production of polymeric scaffolds for tissue growth in vitro or regeneration in vivo presents as a disadvantage low cell adhesion due to the absence of biological recognition. In this context, it is interesting to use synthetic polymers coated with natural polymers, functionalization of the material's surface with bioactive molecules, encapsulation of bioactive molecules favoring cell adhesion, proliferation and migration, essential processes for tissue regeneration. (LUO, ENGELMAYR, et al., 2007). Another interesting strategy consists of the incorporation of bioactive molecules in the synthetic polymer, molecules capable of inducing the cellular behaviors required to promote regenerative processes and also act as anti-inflammatory and antibacterial agents.

The patent document WO 2015021519 protects bioactive vitreous fibers covered by collagen, a natural polymer that gives these fibers a better bio-recognition for biomedical applications.

The addition of natural extracts to biocompatible polymers has been explored with the aim of conferring bio-recognition, in addition to bactericidal, fungicidal and anti-inflammatory properties (ref2-patent attached). The invention protected in WO 2014094085 is chitosan, a natural polymer, in the form of nanoparticles, films, hydrogels and sponges containing an extract of Arrabidaea chica that gives the polymer regenerative properties favoring healing of the skin and mucosa and gastrointestinal tissue.

Polymer composites reinforced with ceramics are an extremely important class of materials for the production of scaffolds used in the regeneration of bone tissue. Bone tissue engineering scaffolds must exhibit sufficient mechanical strength to act as a support while the tissue is regenerated. Polymeric materials exhibit low strength and stiffness and therefore are not compatible with bone tissue, especially when they are in the form of scaffolds with high porosity or in the fibrous form. The strategy to improve the mechanical properties of such polymers consists of using fibers or ceramic particles as reinforcements. Among the ceramic materials that stand out we can mention the bioglass and calcium phosphates which, besides acting as reinforcements, also exhibit bioactivity, an essential property for the regeneration of bone tissue. The use of bioactive molecules with regenerative and antibacterial properties is also of great importance to favor the regeneration of bone tissue and for this reason it has also been added in polymer reinforced with ceramics composites.

The patent WO 2014194392 A1 protects a composite consisting of polyurethane polyol/poly (vinyl alcohol) and hydroxyapatite and applied to the restoration of bone tissue and which exhibits mechanical compression properties suitable for bone grafts of high mechanical demand, as materials for filling the skullcap and bone defects.

Patent document WO 2014016816 protects a polymeric mesh containing bioactive substances used by electrospinning that can be applied in the treatment of diseases that recover or recover the regeneration of the skin, cartilage, periodontal and submucosal ligament of the esophagus. This invention describes or uses synthetic polymers combined with natural polymers or active substances capable of controlling the inflammatory process, the regenerative process and/or homeostasis.

The patent WO 2010121341 refers to a bioactive composite for bone repairs, whose polymeric matrix is composed of polysaccharide and reinforced with calcium-derived compounds. The use of ceramic reinforcements provides bioactivity and mechanical properties suitable for tissue regeneration.

Patent document WO 2013166566 A1 protects a three-dimensional matrix of biodegradable polymer in the form of nanometric and/or micrometric fibers intended for bone, soft and cartilage tissues regeneration, and may contain drugs, ceramics and growth factors, which favor biorecognition and bioactivity.

The invention presented in patent document WO 2013126975 relates to a composite of three-dimensional porous material consisting of bio-absorbable polymers reinforced with bioactive ceramics containing additives capable of facilitating tissue regeneration and formation.

The seed of Bixa orellana L. comes from a native shrub in tropical regions, belonging to the Bixaceae family, a vegetable source of carotenoids, such as bixin and norbixin, widely used as raw material for the production of natural dye for a wide range of manufactured products. The phytochemicals from Bixa orellana L. seeds exhibit pharmacological properties that include, antibacterial, antifungal, antioxidant, anti-inflammatory, antitumor, analgesic and anticonvulsant activity (TAHAM et al., 2015; Barbosa Filho et al., 2014). These components present in Bixa orellana L. make it possible to use its extract in herbal medicines for example, as they promote healing of the epidermis and mucous membranes. (Villar et al., 2014).

### SUMMARY OF THE INVENTION

In the present invention, a new matrix (scaffold) for Tissue Engineering in the form of foams, fibers and/or membranes consisting of polymers, ceramics, polymeric composites and/or ceramic composites containing extract of Bixa orellana L. capable of inducing tissue regeneration in vivo, tissue growth in vitro, avoid inflammatory process and contamination with fungi and bacteria during the regeneration and creation of new tissue. The matrices can be two-dimensional and/or three-dimensional and present morphology, porosity and pore size required for tissue growth and regeneration.

These three-dimensional and/or two-dimensional matrices can be constituted, in a limited way, by polymers, ceramics, drugs, growth factors, in addition to active constituents of natural extracts in the form of foams, fibers and membranes

### BRIEF DESCRIPTION OF THE DRAWINGS

FIG. 1—are SEM images of cellulose acetate nanofibers containing Bixa orellana L. extract.

FIG. 2—are SEM images of PVA nanofibers/Bixa orellana L. extract.

FIG. 3—is an SEM image of the interior of the PVA foam/extract of Bixa orellana L. reticulated with chemical cross-linking agent.

FIG. 4—is an Optical microscopy image of the surface of PVA foams/extract of Bixa orellana L. Reticulated with chemical cross-linking agent.

FIG. 5—is an SEM image of PVA foams/extract of Bixa orellana L. reticulated with chemical cross-linking agent.

FIG. 6—is Poly butylene succinate (PBS) foams/Bixa orellana L. extract.

FIG. 7—is an SEM image of the composite PVA/extract of Bixa orellana L. reinforced with magnetic nanoparticle.

FIG. 8—is an SEM image of the PVA composite/annatto extract Bixa orellana L. reinforced with bioglass.

FIG. 9—is an SEM image of the PVA composite/Bixa orellana L. extract reinforced with bioglass with its respective chemical analysis by EDS.

### DETAILED DESCRIPTION OF THE INVENTION

The present invention relates to a new matrix (scaffold) in the form of foams, fibers and/or membranes consisting of polymers and/or polymer composites reinforced with ceramics containing Bixa orellana L. extract as a bioactive molecule capable to induce tissue regeneration in vivo, tissue growth in vitro and also avoid inflammatory processes and contamination with fungi and bacteria during the regeneration and creation processes of the new tissue. The matrices can be two-dimensional or three-dimensional in the form of foams, fibers and/or membranes and present morphology, porosity and pore size required for tissue growth and regeneration.

In the present invention, biocompatible polymers are considered any material of natural or synthetic origin ordered in the form of chains (dimeric or polymeric) associated in the form of homopolymers, copolymers or polymeric blends, whether of synthetic or natural materials, which do not promote any harmful reaction to the organism or cells and that has degradation due to physiological processes (such as hydrolysis, enzymatic degradation, among others) without generating toxic products.

The natural polymers that can be used to produce scaffolds in the form of foams, fibers and/or membranes that include collagen, gelatin, fibrin, fibronectin, fibrinogen, laminin, proteoglycans, elastin, hyaluronic acid, chondroitin sulfate, polyamino acids, polysaccharides, chitosan , chitin, silk, alginate, modified cellulose, however, any other polymer of vegetable or animal origin can be used.

Biocompatible, biodegradable synthetic polymers that can be used to produce scaffolds in the form of foams, fibers and/or membranes include poly (lactic acid), poly (L-lactic acid) poly (D-Lactic acid), poly (glycolic acid), polycaprolactone, poly (epsilon-caprolactone), poly (lactic-co-glycolic acid), poly (epsilon-caprolactone-co-glycolic acid), poly (epsilon-caprolactone-co-L-lactic acid), polydioxanone, polygluconate , poly (lactic acid-ethylene co-oxide), poly (vinyl alcohol), poly (hydroxybutyrate), poly (hydroxypropionic acid), polyphosphoester, poly (alpha-hydroxy acid), polycarbonates, polyamides, polyanhydrides, polyamino acids, polyorthoesters , biodegradable polyurethanes, cellulose acetate, poly (butylene succinate) and poly (hydroxybutyrate).

The biocompatible polymers of the group of non-degradable polymeric materials that can be used for the production of scaffolds in the form of foams, fibers and/or membranes include aliphatic polyesters, polyacrylates, polymethacrylates, non-degradable polyurethanes, polystyrenes, polyvinyl chloride, polyvinyl fluoride, polyvinyl fluoride imidazole, chlorosulfonated polyolefins, polyethylene oxide, polyvinyl alcohol, polytetrafluoroethylene, polyamides, silicone, poly (styrene-co-butadiene).

The polymer/ceramic composites that can be used to produce scaffolds in the form of foams, fibers and/or membranes include the biocompatible and biodegradable or non-degradable polymers mentioned in the previous paragraphs reinforced with particles or ceramic fibers. In the present invention, ceramic material is any inorganic and non-metallic material, biocompatible, whose synthesis can be carried out by heating at high temperatures or co-precipitation at low temperatures, resulting in a material in the form of crystalline fibers or particles (for example, triphosphate calcium, hydroxyapatite, magnetite, maghemite) or amorphous (for example, glass) that has the ability to induce tissue growth in vitro, tissue regeneration in vivo, direct growth or tissue regeneration and/or induce hyperthermia. Among the ceramic materials used, bioactive and biodegradable calcium phosphates, bioactive and biodegradable ceramics, bio glass, biocompatible iron oxides can be highlighted.

The polymer matrices and polymer/ceramic composites in the form of foams, fibers or membranes contain a concentration of Bixa orellana L. extract that ranges from 0.01% to 50%, but preferably from 0.1% to 25%, in particular from 0.5% to 15% (w/w).

The polymer/ceramic composites containing Bixa orellana L. extract can have a concentration of particles and/or reinforcement fibers in the proportion that varies from 0.01% to 90%, but preferably from 0.1% to 50%, especially of 0.5% to 10% (w/w). The variation in the concentration of reinforcements allows to control mechanical properties, bioactivity, rate of regeneration, growth and direction of growth/tissue regeneration.

The polymeric, ceramic and composites matrices containing Bixa orellana L. extract in the form of foams have different populations of interconnected pores, whose size range varies from 40-900 μm and porosity in the range of 30-90%.

The polymeric, ceramic and composite matrices containing Bixa orellana L. extract in the form of fibers are made up of filaments with an average diameter in the range of 1 nm and 100 μm, with a smooth and/or porous surface.

The polymeric, ceramic and composite matrices containing Bixa orellana L. extract in the form of membranes containing Bixa orellana L. extract with average thickness in the range of 0.1 μm and 10 μm, with smooth and/or rough surface.

The polymeric matrices, ceramics and composites containing Bixa orellana L. extract with concentration in the range of 0.01% to 50% w/w.

Preparation of polymeric matrices, ceramics and composites in the form of foams, fibers, membranes containing Bixa orellana L. extract The processing methods used to obtain the polymer matrix or polymer/ceramic composite containing Bixa orellana L. extract are described below.

Foams—The process of obtaining matrices in the form of foams consists of the combination of methods of particle leaching, foaming and lyophilization. The particle leaching technique involves molding the dissolved or fused polymer around the porogenic agent, drying or solidifying the polymer and leaching the porogenic agent to generate the polymers with an interconnected pore network. The technique produces scaffolds with controlled porosity (over 93%), pore size (over 500 mm) and crystallinity. By adjusting the manufacturing parameters such as type, quantity and size of the porogenic agent, the porous scaffolds can be adapted for a specific application. The Foaming technique uses a chemical reaction whose product is a gas that is trapped in the form of bubbles within the polymeric matrix. The lyophilization technique, on the other hand, consists of producing a polymer solution that is frozen and then subjected to cryogenic cooling, followed by the application of a low pressure to remove ice crystals via sublimation. Lyophilization removes water and solvent, producing the scaffold with interconnected pores and porosity above 90% and an average diameter of 15 to 35 μm.

Fibers—In the present invention, matrices with fibrous morphology are obtained using the electrospinning technique. From this technique, it is possible to form filaments, or fibers, on a micro and nanometric scale. The matrices are prepared by dissolving the polymer in an appropriate solvent. The process takes place through the application of a high voltage between a metallic capillary (needle), connected to a syringe, which contains the polymeric solution and an electrically grounded collector. The solution is ejected through the capillary at a constant rate controlled by an injection pump. When the electric field exceeds the surface tension of the solution, it forms a polymeric jet, which becomes thinner as a result of the evaporation of the solvent, occurring the formation of fibers that are attracted to the collector. The thickness and morphology of the fibers depend on the physical-chemical properties of the solutions, such as viscosity, surface tension, and also on process parameters such as the solution flow, dielectric potential and distance between the needle tip and the collector.

Membranes—The technique known as solvent casting consists of pouring a solution of the polymer into a mold and allowing the solvent to evaporate forming a solid film and film containing the polymeric material in the form of non-porous membranes.

In the present invention, the process of obtaining polymeric matrices or polymer/ceramic composites containing Bixa orellana L. extract in the form of fibers, foams or membranes comprises the following steps:
1—Obtaining Bixa orellana L. extract. This step consists in preparing suspensions of Bixa orellana L. extract in methanol by adding the seeds in organic solvent in the proportion (3:1) (organic solvent/seeds) (m/m) . The suspension is stirred at 55° C. for a period in the range of 10 minutes to 24 hours, then the suspension is filtered, obtaining a suspension of Bixa orellana L. extract in ethanol with a concentration in the range of 1% to 10% m/m.
2—Obtaining the impregnated polymers with Bixa orellana L. extract. The polymeric, ceramic and/or composite mass is mixed with a volume of Bixa orellana L. extract sufficient to obtain the desired proportion of active extract molecules. This mixture is left to stand for evaporation of the solvent. The obtained system is dried obtaining the polymer impregnated with extract of Bixa orellana L. In some variations of the method the extract is added during the processing of the polymeric matrix or polymer/ceramic composite.
3—Processing of polymers, ceramics or polymer/ceramic composites—In this stage, fibers, foams or membranes are produced, using the processing techniques corresponding to each matrix form described above.

### EXAMPLE 1

### Preparation of Cellulose Acetate Fibers Containing Bixa orellana L Extract

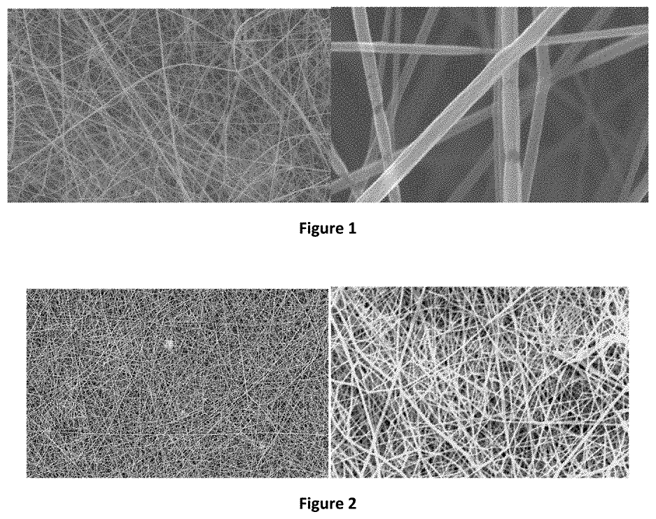

The cellulose acetate solution/Bixa orellana extract with a concentration in the range 1 to 50% w/w was obtained by adding the polymer impregnated with Bixa orellana L., to an acetone /dimethylformamide solution (3:1, v/v) with constant stirring followed until complete solubilization at 40° C. The fibers were obtained by electrospinning the suspension under tension in the range of 5 to 40 kV, in temperature in the range of 5 to 6° C., using a distance in the range of 3 cm to 30 cm between the tip of the needle and the surface of the collector, with flow in the range of 0.005 to 10 mL/h in the pump, and rotation in the range of 1 to 6000 rpm in the collecting cylinder. The SEM images obtained for the cellulose acetate fibers/Bixa orellana L. extract in the different electrospinning conditions are shown in FIGS. 1.

### EXAMPLE 2

### Preparation of Polyvinyl Alcohol Fibers (PVA) Containing Bixa orellana L Extract

The aqueous solution of PVA/Bixa orellana L. extract with concentration 1 to 50% w/w was obtained by adding the PVA impregnated with Bixa orellana L. in ultra pure water under constant agitation at a temperature in the range of 25 to 70° C. by electrospinning the suspension under tension in the range of 5 to 40 kV, in temperature in the range of 5 to 60° C., using a distance in the range of 3 cm to 30 cm between the tip of the needle and the surface of the collector, with flow in the range from 0.005 to 10 mL/h in the pump, and rotation in the range from 1 to 6000 rpm in the collecting cylinder. The SEM images obtained for the PVA fibers/Bixa orellana L. extract in the different electrospinning conditions are shown in FIGS. 2.

### EXAMPLE 3

### Preparation of Polyvinyl Alcohol Foams (PVA) Containing Bixa orellana L. Extract Crosslinked with Glutaraldehyde

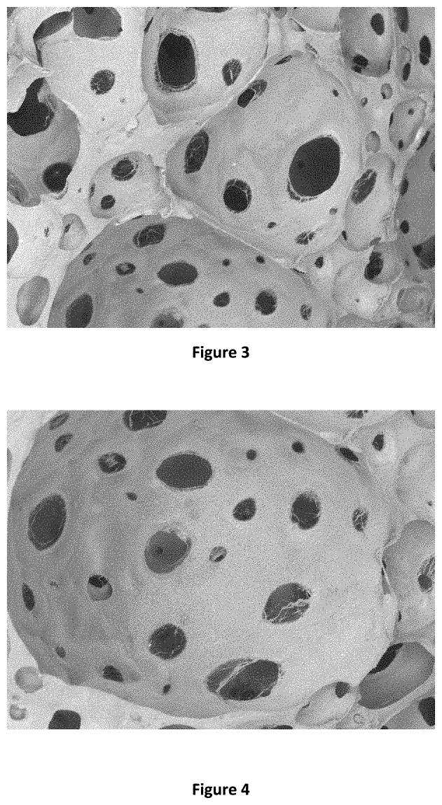

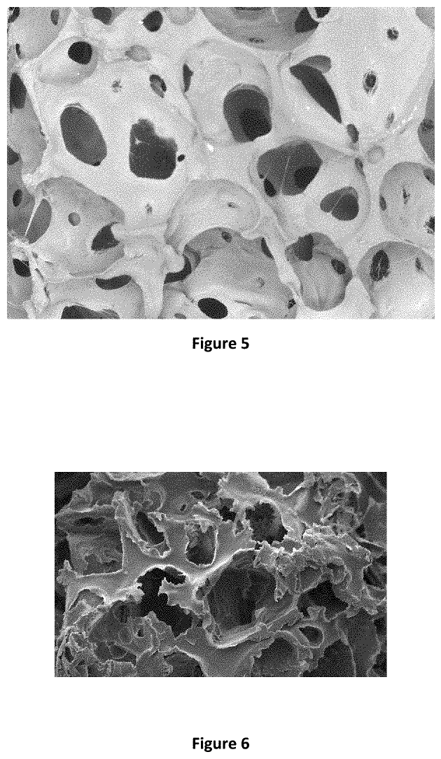

Initially an aqueous suspension of PVA/Bixa orellana L. extract and the porogenic agent was obtained by adding the PVA impregnated with Bixa orellana extract L. and the porogenic agent in ultra pure water under vigorous stirring for 1 minute. This suspension was stirred at a temperature in the range of 50-90° C. The homogeneous solution obtained was cooled to room temperature and then hydrochloric acid was added under gentle stirring. The porous sample obtained was frozen and left to stand at that temperature for 3 days. After this period, the sample was cut into cubes that were dipped in sodium hydroxide solution containing crosslinking agent and kept under agitation. The samples obtained were cooled and left to rest. Then, they were washed to leach the residual salt. The samples were frozen in liquid nitrogen and lyophilized. The SEM images of the porous foams of PVA/extract of Bixa orellana L. obtained are shown in FIGS. 3, 4 and 5.

### EXAMPLE 4

### Preparation of Polyvinyl Alcohol Foams (PVA) Containing Physically Crosslinked Bixa orellana L.

Initially, an aqueous suspension of PVA/Bixa orellana L. extract and the porogenic agent was obtained by adding the PVA impregnated with Bixa orellana L. extract and the porogenic agent in ultra pure water under vigorous stirring for 1 minute. This suspension was stirred at a temperature in the range of 50-90° C. The homogeneous solution obtained was cooled to room temperature and then hydrochloric acid was added under gentle stirring. The porous sample obtained was frozen and left to stand for 3 days. After this period, the sample was cut into cubes that were subjected to 5 cycles of thawing at room temperature, followed by freezing. Then, they were washed to leach the residual salt. The samples were frozen in liquid nitrogen and lyophilized.

### EXAMPLE 5

### Preparation of Poly Butylene Succinate (PBS) Foams Containing Bixa orellana L. Extract

The PBS containing Bixa orellana L. extract was dissolved in organic solvent, the solution obtained was poured onto a flat surface containing a layer of porogen agent. The obtained system was left to stand at room temperature and after complete evaporation of the solvent the sample was washed several times until the agent was completely leached. The SEM images of the porous foams of PBS/Bixa orellana L. extract obtained are shown in FIG. 6.

### EXAMPLE 6

### Preparation of Polymeric Polyurethane (PU) Membranes Containing Bixa orellana L. Extract

The PU impregnated with Bixa orellana L. extract was dissolved in dimethylformamide (DMF) to obtain a concentration solution in the range of 1 to 30% m/v. The mixture was stirred for 24 hours at a temperature in the range of 30° C.-50° C., until complete PU solubilization. Subsequently, the solution was poured on a surface, the evaporation of the solvent occurred at a temperature in the range of 30° C.-50° C., for 24 hours.

### EXAMPLE 7

### Preparation of PVA Composite/Bixa orellana L. Extract Reinforced with Nanoparticulate Magnetite

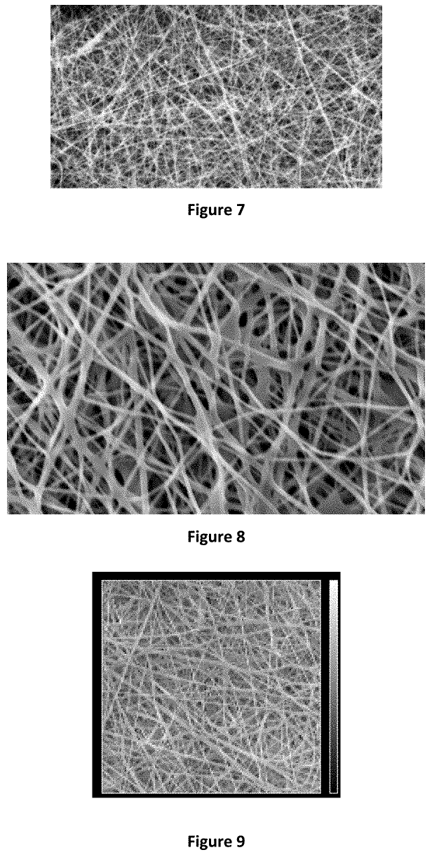

The aqueous solution of PVA/Bixa orellana L. extract with concentration 1 to 50% w/w was obtained by adding the PVA impregnated with extract in a aqueous suspension of nanoparticulate magnetite with concentration in the range of 0.5 to 30% m/m under constant agitation at temperatures in the range of 30° C.-50° C. The composites in the form of fibers reinforced with magnetic nanoparticles were obtained by electrospinning the suspension under tension in the range of 5 to 40 kV, in temperature in the range of 5 to 60° C., using a distance in the range of 3 cm to 30 cm between the tip of the needle and the surface of the collector, with flow in the range of 0.005 at 10 mL/h in the pump, and rotation in the range of 1 to 6000 rpm in the collecting cylinder. The SEM images obtained for the composite are shown in FIGS. 7.

### EXAMPLE 8

### Preparation of PVA Composite/Bixa orellana L. Extract Reinforced with Bioactive Glass

The aqueous solution of PVA/Bixa orellana L. extract with concentration 1 to 50% w/w was obtained by adding the PVA impregnated with Bixa extract orellana L. in a bioactive glass suspension with a concentration of 0.5 to 30% w/w under constant agitation, temperatures in the range of 30° C.-50° C., followed by sonication. Composites in the form of fibers reinforced with bioglass particles were obtained by electrospinning the suspension under tension in the range of 5 to 40 kV, in temperature in the range of 5 to 60° C., using a distance in the range of 3 cm to 30 cm between the tip of the needle and the surface of the collector, with flow in the range of 0.005 to 10 mL/h in the pump, and rotation in the range of 1 to 6000 rpm in the collection cylinder. The SEM images obtained for the composite are shown in FIGS. 8 and 9.

Although the preferred version of the invention has been illustrated and described, it should be understood that it is not limited. Various modifications, changes, variations, substitutions and equivalents may occur, without departing from the scope of the present invention.

1. Matrices for Tissue Engineering in the form of foams, fibers and/or membranes consisting of polymers, ceramics, polymeric composites and/or ceramic composites characterized by containing an active substance, the extract of Bixa orellana L., with concentration in the range of 0.01% to 50% w w; and the method of obtaining it.

2. Matrices for Tissue Engineering in the form of foams, fibers and/or membranes consisting of polymers, ceramics, polymeric composites and/or ceramic composites as described in claim 1. is characterized by inducing tissue regeneration in vivo, tissue growth in vitro, and avoiding inflammatory processes and contamination with fungi and bacteria during the processes of regeneration and growth of new tissues.

3. The Matrices for Tissue Engineering according to claim 1 characterized by a material in the form of foams consisting of structures containing with open and interconnected pores with size in the range of 40-900 μm and porosity in the range of 30-90%.

4. The Matrices for Tissue Engineering according to claim 1 characterized by a material in the form of lined or randomly oriented fiber mats consisting of filaments with average diameters in the range of 1 nm and 100 μm, with smooth, rough and/or porous surface.

5. The Matrices for Tissue Engineering according to claim 1 characterized by a material in the form of membranes containing pores or not, with an average thickness in the range of 0.1 μm and 10 μm, with smooth and/or rough surface.

6. The Matrices for Tissue Engineering according to claims 1 to 5, characterized by a biocompatible material from the group of polymeric, ceramic and/or composite materials containing the active substance.

7. The Matrices for Tissue Engineering, according to claim 6, characterized by a biocompatible material from the group of natural and/or synthetic biodegradable polymeric materials, as well as the combination of these in the form of copolymers and/or blends, containing the active substance.

8. The Matrices for tissue engineering, according to claim 6, characterized by a material from the group of non-degradable polymeric materials, containing the active substance.

9. The Matrices for Tissue Engineering, according to claim 6, characterized by a biocompatible material of the class of ceramic materials containing the active substance.

10. The Matrices for Tissue Engineering, according to claim 6, characterized by a biocompatible material from the group of composite materials consisting of polymeric materials, according to claims 7 and 8, and/or ceramic materials, according to claim 9, reinforced with particles and/or fibers, according to claims 7-9, added in a concentration ranging from 0.01% to 70% w/w.

11. Method of obtaining biocompatible material from the group of polymeric, ceramic and composite materials containing the active substance as defined in claims 6 to 10, characterized by the following steps:
a. Impregnation of polymeric, ceramic and composite materials with the active substance;
b. Drying of the material containing the active substance;

12. Method for obtaining biocompatible material from the group of polymeric, ceramic and composite materials containing the active substance, according to claim 11 (a), characterized by adding the solution in organic solvent of the active substance, with a concentration in the range of 0.005% to 50% w/w, over a period of time from 5 minutes to 48 hours to materials in the polymer class, according to claims 7 and 8 and/or materials in the ceramic class, according to claim 9, in powder, extract or grains.

13. Method for obtaining biocompatible material from the group of polymeric, ceramic and composite materials containing the active substance, according to claim 11 (b), characterized by drying the material incorporated with the substance by increasing the temperature, in the range of 30-200° C., pressure reduction and lowering of temperature, use of supercritical fluid and/or application of vacuum with or without temperature change.

14. Method of obtaining the biocompatible polymeric material as defined in claims 1 and 6 characterized by:
a. processing the matrices in the form of foams;
b. processing the matrices in the form of fibers;
c. processing the matrices in the form of membranes;

15. Method of biocompatible material from the group of polymeric, ceramic and composite materials in the form of foams, according to claim 14 (a), characterized by
(i) Addition of the porogenic agent to a polymeric mass and/or ceramic mass in suspension or not, in proportions from 1:1 to 20:1 w/w material/porogenic agent, the porogenic agent being selected from the group of: particles soluble in aqueous solution, frozen or effervescent particles and/or droplets of solvents insoluble in the dispersion of the polymeric material or ceramic material, particles obtained from the gas product formation reaction, or gas foams;
(ii) Transfer of the polymeric mass and/or ceramic mass in suspension or not containing the porogenic agent to mold and promote drying, temperature increase, in the range of 30-200° C., pressure reduction and lowering of temperature or application of vacuum with or without temperature change.
(iii) removal of the porosity-forming agent through leaching, chemical reaction, porogenic fusion, sintering, pressure reduction and porogenic dissolution.

16. Method of biocompatible material from the group of polymeric materials and composites in the form of fibers, according to claim 14 (b), characterized by including the methods for producing non-woven mats of aligned or with random orientation that includes the following steps:
(i) dissolving the polymer impregnated with the active substance in the corresponding solvent, obtaining a solution with a concentration between 5 and 35% w/w, temperatures from 25 to 150° C.;
(ii) electrospinning of the solutions, c)nsisting of a mono or coaxial electrospinning process, characterized by the electrospinning conditions of the nanofibers: voltage in the range of 5 to 40 kV, temperature in the range of 5 to 60° C., using a distance between the tip of the needle and the surface of the collector in the range of 3 cm and 30 cm, with flow in the pump in the range of 0.005 to 10 mL/h and rotation in the range of 1 to 6000 rpm in the collection cylinder.

17. Method of biocompatible material from the group of polymeric and composite materials in the form of membranes, according to claim 14 (c), characterized by obtaining the membranes by the solvent casting process that includes the preparation of the solutions of the polymeric materials and/or composites, according to claims 7, 8 and 10, with concentration in the range of 5 to 90% w/w, temperature in the range of 25 to 150° C., followed by spreading the solution on a flat surface and drying in temperature in the range from 25 to 200° C.
